(19) United States
(12) Patent Application Publication
(10) Pub. No.: US 2006/0084953 A1
(43) Pub. Date: Apr. 20, 2006

(54) Multibeam laser for skin treatment

(76) Inventor: Nikolai Tankovich, San Diego, CA (US)

Correspondence Address: JOHN R. ROSS, PO BOX 2138, DEL MAR, CA, 92014, US

(21) Appl. No.: 11/195,511

(22) Filed: Aug. 1, 2005

Related U.S. Application Data
(60) Provisional application No. 60/598,201, filed on Aug. 2, 2004.

(51) Int. Cl. A61B 18/18

(52) U.S. Cl. 606/9; 606/3; 606/10; 606/13

(57) ABSTRACT

A skin treatment laser device suitable for home use. The device includes an array of at least two types of laser units. A first type of laser units operates at a wavelength chosen to heat the skin generally and a second type of laser units operates at a wavelength chosen to produce tissue damage in extremely small and separated regions of the skin. No skin damage occurs in tissue surrounding the regions of skin transformation or destruction. Natural healing processes originating in the surrounding tissue heal the damaged tissue and produce general rejuvenation effects in the skin tissue. Laser beams from at least the second type of laser units are focused into the small regions to be damaged. A preferred focusing technique utilizes cold sapphire cylindrical rods. Preferably, the skin to be treated is shaved or abraded prior to or simultaneously with the laser treatment to remove portion of the stratum corneum so to improve laser beam penetration. In preferred embodiments the device includes shaving blades so that skin hair and or a thin layer of skin can be removed simultaneously with the laser treatment. In other embodiments an abrading feature is included.

This invention relates to lasers and in particular to lasers for cosmetic treatments. This application claims the benefit of Provisional Application Ser. No. 60/598,201 filed Aug. 2, 2004.

### BACKGROUND OF THE INVENTION

Use of lasers for medical purposes is well established. Lasers are used extensively for purposes such as hair removal, vein treatment, skin rejuvenation and treatment of port wine stain. Each of these treatments is preferably performed by medical practitioners with a laser producing laser pulses at a wavelength chosen to be most effective for the particular treatment. Some wavelengths are very preferentially absorbed in a particular type of tissue. Some wavelengths are highly absorbed in skin tissue with penetration depths of only a few microns. Other wavelengths have absorption coefficients substantially less than 1/cm and penetrate substantial depths in skin and other tissue. FIGS. 4 and 5 show absorption coefficients as a function of wavelengths for blood, human skin and melanin. A Nd:YAG laser operating at 1320 nm (with high absorption in skin tissue) may be used for skin rejuvenation and micro skin surgery. Treatment of port wine stains is usually performed using a dye laser operating at a wavelength of 577 nm and 585 nm where the absorption in blood hemoglobin is high but absorptionin the skin tissue is relatively low.

Use of a laser beam matched to a peak or relatively high absorption in tissue to treat the tissue is referred to as “selective thermolysis”. When wavelengths which penetrate deeply and are absorbed relatively uniformly in tissue are used to treat the tissue, the treatment is referred to as “non-selective thermolysis”.

Beautiful young looking skin is very important to most people, especially women. They spend billions of dollars each year in their efforts of look their best. Skin treatments often provide only temporary help and need to be repeated. Laser treatments at medical and cosmetic facilities are expensive.

What is needed is a skin treatment laser device suitable for home use.

### SUMMARY OF THE INVENTION

The present invention provides a skin treatment laser device suitable for home use. The device includes an array of at least two types of laser units. A first type of laser units operates at a wavelength chosen to heat the skin generally and a second type of laser units operates at a wavelength chosen to produce tissue damage in extremely small and separated regions of the skin. No skin damage occurs in tissue surrounding the regions of skin transformation or destruction. Natural healing processes originating in the surrounding tissue heal the damaged tissue and produce general rejuvenation effects in the skin tissue. Laser beams from at least the second type of laser units are focused into the small regions to be damaged. A preferred focusing technique utilizes cold sapphire cylindrical rods. Preferably, the skin to be treated is shaved or abraded prior to or simultaneously with the laser treatment to remove portion of the stratum corneum so to improve laser beam penetration. In preferred embodiments the device includes shaving blades so that skin hair and or a thin layer of skin can be removed simultaneously with the laser treatment. In other embodiments an abrading feature is included.

### BRIEF DESCRIPTION OF THE DRAWINGS

FIGS. 1A and 1B are drawings of a preferred embodiment of the present invention.

FIGS. 1C and 1D shows beam paths into the skin and features for removing a portion of the stratum corneum.

FIGS. 2 and 3 are drawings showing human skin features.

### DETAILED DESCRIPTION OF PREFERRED EMBODIMENTS

Preferred embodiments of the present invention may be described by reference to the drawings.

### First Preferred Embodiment

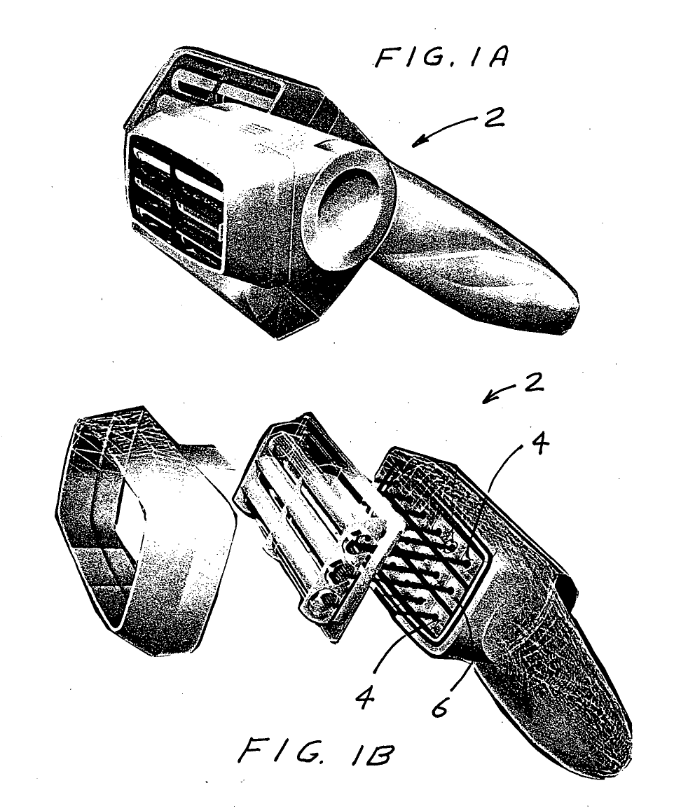

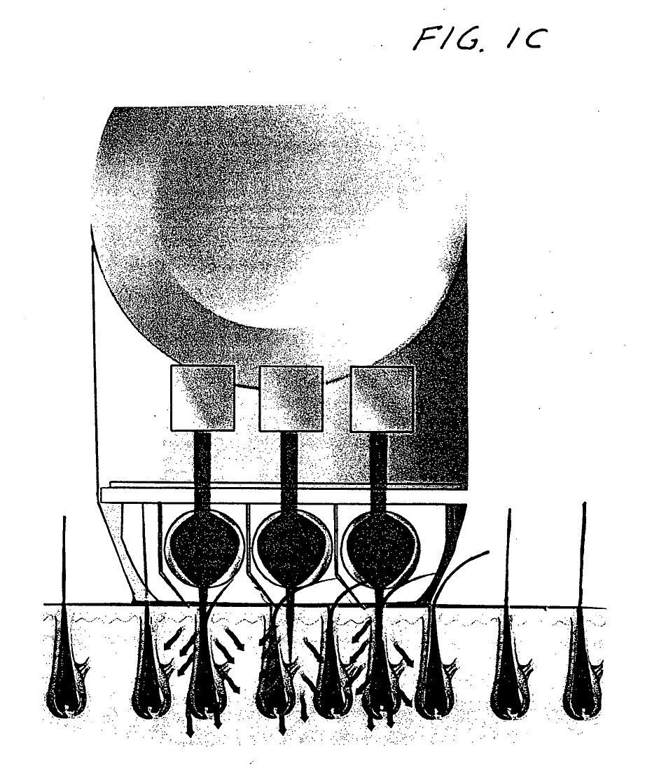

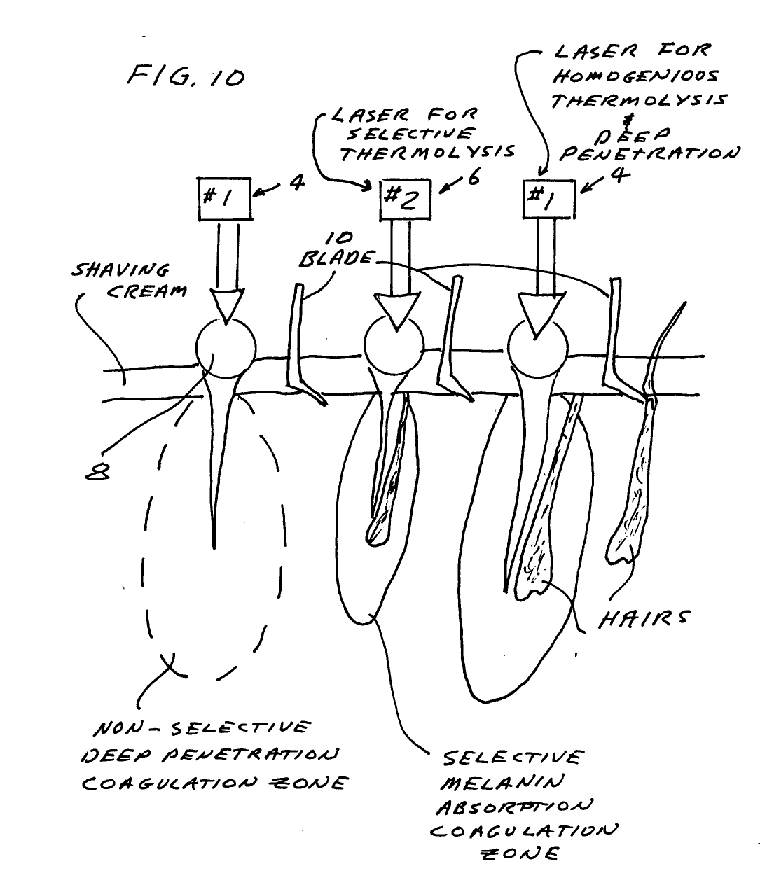

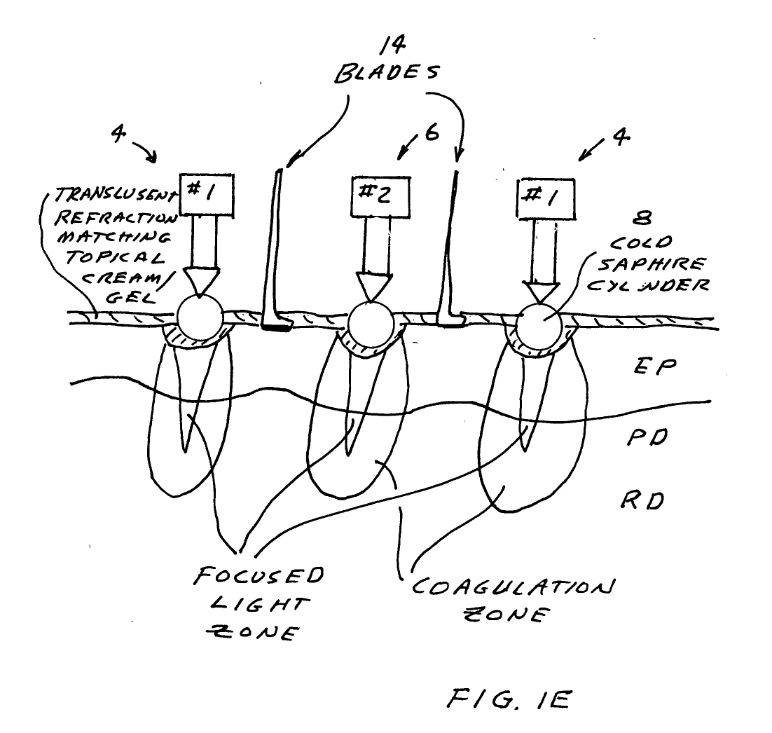

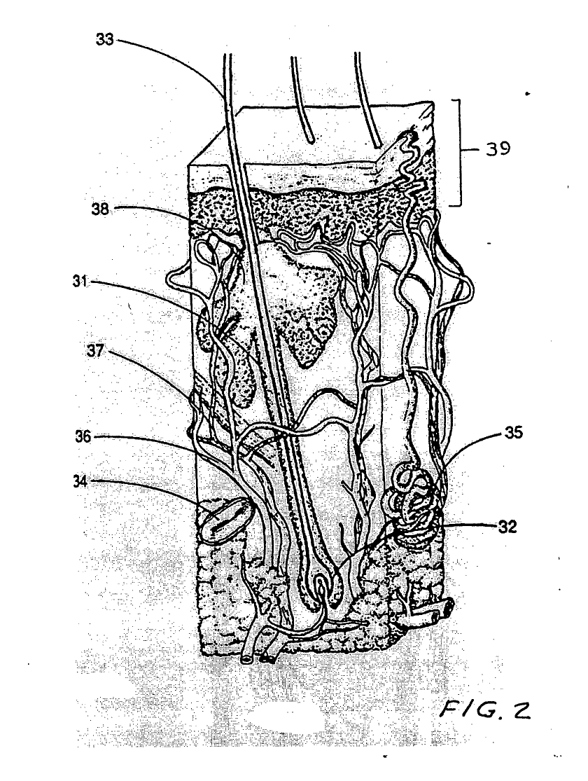

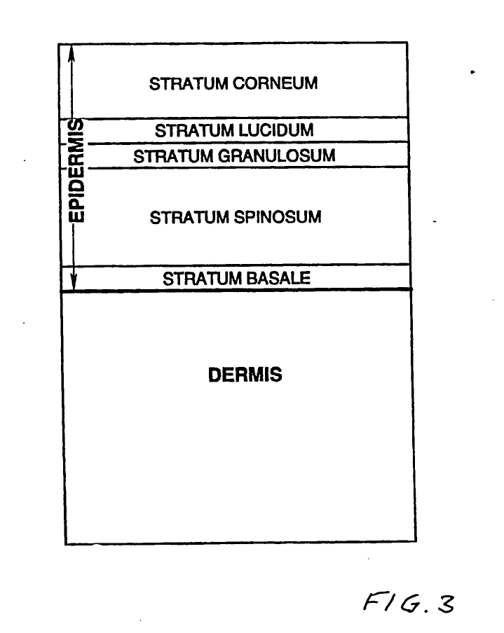

A first preferred embodiment of the present invention may be described by reference to FIGS. 1A, 1B, 1C and 1D. This device 2 includes twelve diode lasers 4 operating at a wavelength chosen to heat the skin generally to temperatures a few degrees below temperatures at which damage begins to occur at depths down to about 2 millimeters. Device 2 also includes six diode lasers 6 operating at wavelengths chosen to produce skin damage in very small regions of the skin. The device also includes three cylindrical sapphire focusing lenses 8 aligned with the lasers as shown in FIG. 1B, 1C and 1D. Light from diode lasers 6 is tightly focused into a small region of the skin since at wavelengths of lasers 6 is very absorptive in skin and produces relatively little scattering. However, light from lasers 4 are not focused tightly by lenses 8 since light at its wavelengths are scattered widely by skin tissue. This first preferred embodiment also includes blades 10 for cutting hair on the skin. Preferably the blades are replaceable. The blades serve two purposes (1) they remove any hair at the skin surface and (2) they remove a portion to the stratum corneum which improves the transmission of the laser beams into the skin. Preferably, a translucent refractive index matching shaving cream is used as shown at 12 in FIG. 1D to make shaving easier and also improve beam penetration.

### Second Preferred Embodiment

A second preferred embodiment is shown in FIG. 1E. This embodiment is very similar to the first embodiment except shaving blades 10 are replaced by skin abrading blades 14.

### Third Preferred Embodiment

A known technique for subsurface collagen remodeling using a Perovskite 1341 nm laser involves a space partial thermolysis of papillary and reticular dermis to produce a high absorption of IR light in upper layers of the skin. Its multi beam output at 1341 nm is combined with the output of a YAPerovskite:Nd, 1079 nm laser using multibeams combining optical fibers connected to a hand piece.

The YAPerovskite:Nd laser at 1079 nm is used to improve selective thermolysis at 1341 nm by using non-selective thermolysis at 1079 nm. The 1079 nm laser light of YAP:Nd laser has no specific absorption in epidermis or dermis of the skin. The 1079 nm light is very highly scattered in skin and is fairly uniformly absorbed down to depths of a few millimeters. The 1079 nm light thus heats skin though all layers of skin creating a heated epidermis, dermis and subcutaneous fat. This enhanced heating when added to the heating produced in the upper dermis enhances a tissue transformation and modification effect produced by the 1341 nm beam alone. Therefore care must be taken to be certain that excessive transformation or destruction of the surrounding skin does not occur. To prevent an excessive damage and to control a tissue transformation process a ratio between fluences of 1079 and 1341 can be changed by allowing one or another light to transmit more or to dump more when passing through the optics. A ratio between irradiated and not irradiated skin patterns can be variable as well to allow a bigger portion of the non-treated skin to provide cooling and then healing the effected by a light skin portions. Tissue destruction generally depends on temperature and time. Therefore care should be taken to apply the laser energy at rates will produce only minimal damage to tissue which is not targeted. A technique to keep the temperature of non-targeted tissue below the damage threshold is to cool it with sapphire cylindrical lenses that are cooling elements at the same time. Conductive cooling of the skin surface is desired before, during and after the laser treatment. A set of diode lasers are placed at the first row of the device. Diode lasers emit energy at 720 nm to be well absorbed by a topical gel containing a chromophore Indocyanin Green ICG, glycolic acid and Glycerin. A role of the topical gel with glycolic acid and chromophore ICG to partially destroy a stratum corneum of the epidermis by interacting with a diode light (ICG). Glycerin role is to transmit more light through the stratum corneum by filling a space between dry layers of the upper skin. A handpiece contains two rows of stratum corneum removal blade located between cold cylindrical lenses. Stratum corneum is partially or totally removed by blades after the destruction by glycolic acid, ICG and diode light. Stratum corneum blade has a sharp cutting and scrabbing edge of 150 micron. A pressure applied to the blade will not reach blood vessels during stratum corneum removal not causing any bleeding. Removal of stratum corneum allows an improving significantly a transdermal delivery properties of the upper skin as well as optical properties. Stratum corneum shaving blade can be used before, after during or separately to deliver cosmoceuticals, medications, proteins and other substances into or through the skin. A preferred technique is to apply 8 beams in bursts of pulses with about 10 pulses in two seconds so that the total energy deposited in the skin is about 12 Joules/cm2 if surface cooling is not used. With surface cooling the energy deposited could be increased to about 40 Joules/cm2. Preferably, the ratio of the 1079 nm energy to the 1341 nm energy is about 2 to 1.

### Components

Diode 720 nm laser is available from Asah Corporation with office in Copenhagen, Denmark. Optical fiber with multiple inputs and one output is available from Newport Corporation, Irvine, Calif. The YAP:Nd laser is available from Fotona d.d. with offices in Ljubljana, Slovenia.

### Other Similar Embodiments

Nd:YAG laser in IR range 1320 nm and diode laser at 1450 nm might be also improved by using YAP:Nd as a second preferred embodiment. Nd:YAG laser deck is available from Sciton with office in Palo Alto, Calif. Diode laser 1450 is available from Candela, Wayland, Mass.

### Fourth Preferred Embodiment

### YAP Enhancer for 1.5 Diode or Fiber Laser

A well known technique for wrinkle removal and treating pigmented lesions and acnes on the skin involves the use of lasers at 1.5 um. This technique is based on the fact that 1.5 um laser light is absorbed by the upper layers of the skin that leads to coagulation of those tissues. However, light at this wavelength does not penetrate skin tissue very well and as a result it is difficult to target tissues deeper than 700-800 um without seriously damaging the non-targeted skin tissue. Also this type of laser is difficult to use on a darker type of skin because of higher risk to damage skin.

In an alternate embodiment InP diode 1.5 um laser beams are enhanced with the output of a YAP:Nd, 1079 nm laser. The YAP:Nd 1079 nm laser does not have specific high absorption in skin and the laser light penetrates much deeper in skin than the light of 1.5 um. With the help of 1079 nm light it is easy to provide sufficient energy deposition at deeper layers of skin where the target is located. This combination increases the effect of 1.5 um radiation to the deeper layers of the skin to extend from 700-800 um up to 2-2.5 mm.

Fiber lasers are available from IPG Photonics, Oxford, Mass. InP diodes are available from Covega, Jessap, Md.

### YAP Enhancer for Diode Laser

YAP lasers can be used to enhance performance of diode lasers using the same approach as described above. Medical laser diode laser might be available from Coherent, Santa Clara, Calif., model LightSheer, DioMed, Boston, Mass., Both lasers operate at 800-810 nm range and might be improved by both 1079 and 1341 nm YAP:Nd laser. The 1079 nm wavelength of YAP:Nd laser improves hair removal using diodes lasers, while 1340 nm line of YAP:Nd improves coagulation of small blood vessel by non-selective thermolysis.

All variety of laser diodes in wavelength range 650-1550 are available from SLI Corporation Binghamton, N.Y. YAP laser might be located at the same platform as the diode laser. Optical fiber with two inputs and one output is available from Newport Cortoration, Irvine, Calif.

### Fifth Preferred Embodiment

YAG:Er at 2936 nm performs surface wrinkle (superficial) ablation. The effect is based on the very high absorption of YAG:Er laser light in water. This laser works pretty well on small surface wrinkle but does not remove large deep wrinkles because the 2936 nm beam is extremely well absorbed in skin tissue so the penetration is only a few microns. The YAP:Nd laser at 1341 nm will penetrate a few milimeters and provides subsurface wrinkle treatment. Thus, YAP:Nd laser may preferably be used to enhance YAG:Er laser to perform both surface and deep wrinkles treatment. The approach is similar to those discussed above except in this case the beams are combined in an articulated arm 20 instead of the fiber optic. YAG:Er crystal might be obtained from -Litton Airtron with office in Charlotte, N.C. YAG:Er lasers deck is available from Continuum with office in Santa Clara, Calif., Focus Medical, Bethel, Conn., Fotona, Ljubljana, Slovenia. Articulated arm is available from MedArt Technology, San Diego, Calif.

### Simultaneous Application

Both beams should preferably be applied during the same time interval. The beams may be but do not have to be synchronized. There should not be an significant delay in applying YAG:Er and YAP:Nd laser pulses, since the treated tissue may start to swell very soon after treatment which results in dramatic change in its optical and physiological properties.

### Other Embodiments

Other preferred embodiments include other Nd contained laser crystals such as GGG, GSGG, YAG at around 1320 nm or another Er contained crystal YSGG at 2791 nm.

### Optical Components

The various optical components needed to fabricate the laser system described above are available from normal optics suppliers and techniques for arranging the components are well known to persons skilled in the laser-optics art. For example the YAP:Nd rods for production of the 1079 nm and 1341 nm beams are available from Crytur, Ltd. with offices in Palackeho175, 51101 Tumov, Czeck Republec and Scientific Material Corp. with offices in Bozeman, Mont. Optics for arranging the resonator cavities are available from CVI Corp. with offices in Albuquerque, N. Mex. Flash lamp pumps for these crystal rods are available from Perkin Elmer with offices in Sunnyvale, Calif. Mirrors 12, 20, 22, and 24 and the optics shown to combine both laser lights are available from CVI Corp.

### Preferred Specifications

The power supplies, pump sources and crystal rods should be sized to pulse energies appropriate for the particular treatments planned. In general pulse energies of about 20 J per pulse for the selective beam and about 20 J per pulse for the transmissive beam is recommended. The beam diameters prior to coupling into the optical fiber optic is about 3 mm or more. The beams are normally focused onto the skin surface to produce fluences in the range of about 30 to 90 J per cm² during the treatment period. Fluencies in excess of 50 J per cm² could cause severe skin damage. However, as explained above damage can be avoided or minimized with prior, simultaneous or immediately subsequent cooling.

Although the present invention has been described in terms of preferred embodiments the reader should understand many changes and additions could be made without changing the nature of the invention. Therefore, the scope of the invention is to be determined by the appended claims and their legal equivalents.

What is claimed is:

1. A medical-cosmetic laser system comprising:
A) a plurality of first lasers each laser having a first gain medium and a first set of laser optics configured to produce from said first gain media a plurality of first laser beams defining a first absorption coefficient in typical human skin tissue at a first wavelength preferably absorbed in a target tissue, and
B) a plurality of second lasers each laser having second gain medium and a second set of laser optics configured to produce from said second gain medium a plurality of second laser beams at a second wavelength having an absorption coefficient in human skin defining a second absorption coefficient at least 10 times lower than said first absorption coefficient.
C) a combining means for combining said plurality of first and said plurality of second laser beams to produce a combined laser beam and
D) a means for applying said combined beams to tissue for medical or cosmetic treatments.

2. A laser system as in claim 1 wherein said first wavelength is about 532 nm and said second wavelength is about 1079 nm.

3. A laser system as in claim 2 wherein said first gain medium comprises a Nd:YAG crystal and a said first set of laser optics comprises a frequency doubling KTP crystal.

4. A laser system as in claim 3 wherein said second gain medium comprises a YAP:Nd crystal and said second set of laser optics are configured to produce 1079 nm laser beams from said YAP:Nd crystal.

5. A laser system as in claim 1 wherein said first gain medium and said first set of laser optics is configured as a dye laser capable of producing light at at least one wavelength rang in the wavelength range of 550 nm to 585 nm.

6. A laser system as in claim 1 wherein said first gain medium and said first set of laser optics define a diode laser and said first wavelength is a wavelength in the wavelength range of about 800 nm to 810 nm.

7. A laser system as in claim 6 wherein said second wavelength is about 1079 nm.

8. A laser system as in claim 6 wherein said second wavelength is about 1340 nn.

9. A method of treatment comprising the steps of combining a first laser beam defining a first wavelength having a high first absorption coefficient in a target tissue and defining a first absorption coefficient in typical human skin tissue, with a second laser beam defining a second wavelength having an absorption coefficient in said typical human skin tissue at least 10 times lower than said first absorption coefficient to produce a combined laser beam and applying said combined laser beam to tissue for selective destruction of a target tissue.

10. A laser system as in claim 9 wherein said first wavelength is about 532 nm and said second wavelength is about 1079 nm.

11. A laser system as in claim 10 wherein said said first laser beam is produced in first laser system comprising a Nd:YAG crystal and a frequency doubling KTP crystal.

12. A laser system as in claim 11 wherein said second laser beam is produced in a laser comprising a YAP:Nd crystal and said second set of laser optics are configured to produce 1079 nm laser beams from said YAP:Nd crystal.

13. A laser system as in claim 9 wherein said first laser beam is produced in a dye laser capable of producing light at at least one wavelength rang in the wavelength range of 550 nm to 585 nm.

14. A laser system as in claim 9 wherein said first laser beam is produced in a diode laser and said first wavelength is a wavelength in the wavelength range of about 800 nm to 810 nm.

15. A laser system as in claim 14 wherein said second wavelength is about 1079 nm.

16. A laser system as in claim 14 wherein said second wavelength is about 1340 nm.

17. A laser system as in claim 1 and further comprising a cutting means for removing a portion of skin stratum corneum.

18. A laser system as in claim 1 and further comprising a cutting means for removing a portion of skin stratum corneum and skin hair.
